(19) United States
(12) Patent Application Publication
(10) Pub. No.: US 2021/0319875 A1
(43) Pub. Date: Oct. 14, 2021

(54) STRESS MANAGEMENT SYSTEM

(71) Applicant: DEMAND CO.,LTD, Gyeonggi-do (KR)

(72) Inventors: Gwangsoon KIM, Gyeonggi-do (KR); Jaehoon KIM, Gyeonggi-do (KR); Hannuri PARK, Gyeonggi-do (KR); Youngje JEON, Seoul (KR)

(21) Appl. No.: 17/102,692

(22) Filed: Nov. 24, 2020

(30) Foreign Application Priority Data
Apr. 10, 2020 (KR) 10-2020-0043845

(51) Int. Cl. G16H 20/70; A61M 21/02; G16H 40/60

(52) U.S. Cl. CPC G16H 20/70; A61M 21/02; G16H 40/60; A61M 2021/0027; A61M 2230/04; A61M 2230/06; A61M 2205/505

(57) ABSTRACT

The present invention relates to a stress management system which more accurately calculates a stress index by considering both an objective index through measurement of a pulse wave of a user and a subjective index using a psychological test and an emotion index and provides multiple contents capable of alleviating the resulting stress of the user.

### TECHNICAL FIELD

The present invention relates to a stress management system, and more particularly, to a stress management system which more accurately calculates a stress index by considering both an objective index through measurement of a pulse wave of a user and a subjective index using a psychological test and an emotion index and provides multiple contents capable of alleviating the resulting stress of the user.

### BACKGROUND ART

Most people live under persistent stress for personal reasons, relationships, social issues, etc., and such stress is known as one of the biggest threats to health.

In particular, while the number of emotional labors increases, which control emotions thereof and express the emotions required systematically in the process of performing work, more and more workers suffer from mental health problems such as depression and anxiety stress due to the emotional labor.

Regarding the working conditions and the degree of emotional labor performance, two-thirds of respondents complained that the respondents had stress due to excessive and unreasonable demands in customer service, and there is no support system or protection system when problems arise during the customer service process, and as a result, it was found that the respondents felt that management or measures were not being implemented. Half of the respondents responded that “the organization monitors the work of emotional workers and feels that they interfere too much”, and it was found that the emotional damage caused by the emotional labor was large, and the emotions remained unrecovered.

Regarding job stress, 11.1% of men and 17.1% of women were high-risk groups. Regarding depression, 44.1% of all respondents said they had a feeling of depression, which was far higher than the prevalence of depression (5 to 6%) in the general population. Regarding anxiety, 35.5% of all respondents answered that they had experienced anxiety, and 18.2% of all respondents were at high risk of suicide.

As such, there have been attempts to reduce stress by measuring stress, which is a great risk factor for mental health.

Korean Patent Registration No. 10-1223889 discloses Apparatus and Method for Analyzing of Stress which measure the stress index from a pulse wave signal and then provide audio (music) in order to reduce the stress index. However, Korean Patent Registration No. 10-1223889 has a psychological stability or calming effect of anxiety through audio (music) or breathing, but there is a limit to improving the active mental response ability of the user. In addition, the measurement of stress through pulse waves in Korean Patent No. 10-1223889 has a limitation in accuracy because the stress is measured by using the average of each age and sex without taking into account the bio-signals that vary according to the user's physical characteristics and circumstances. Further, the Korean Patent Registration No. 10-1223889 does not mention that the emotional state can be continuously monitored with the consent of the emotional worker (call center staff), and that the stress state can be alleviated and managed before reaching a dangerous state.

### DISCLOSURE

### Technical Problem

The present invention provides a stress management system that improves not only a quick psychological stability or calming effect, but also a user's active mental response ability.

The present invention provides a system for measuring and managing a stress index in consideration of all subjective emotions such as mental state as well as a user's pulse wave.

The present invention provides a system capable of continuously monitoring the psychological state of users (including emotional workers) and mitigating and managing stress before reaching a dangerous state.

### Technical Solution

In an aspect, the present invention relates to a stress management system including:

a user terminal downloading and executing an in-mind application, detecting a pulse wave signal with a camera sensor and an in-built flash and transmitting the detected pulse wave signal to the server, receiving a stress index and a recommended content corresponding thereto from the server, and executing the recommended content in the application; and

the in-mind server receiving digital data of the pulse wave signal from the user terminal and calculating heart rate variability, automatic nerve activity, and a pulse wave stress index and providing the pulse wave stress index and the recommended content corresponding thereto to the user terminal,

in which the server determines the pulse wave stress index as the stress index,

the recommended content includes healing sounds, a meditation method, a breathing method, and an emotion diary,

the user terminal executes a three-line diary item among the emotion diary contents, selects one or more of a plurality of emotion items for today's emotional state, and then records the selected emotion item, and executes a rethinking module in the emotion diary content and selects one or more of a plurality of emotion items for an emotion state which annoys the user terminal and then, records the selected emotion item, and

the server stores a result of the emotion state selected and recorded by the user terminal in a DB and monitors the result.

### Advantageous Effects

The system of the present invention provides a screen which can not only provide content (passive content) that listens to music or nature, but also records the situation or emotion that annoys the user, and then rethink the recorded situation or emotion from the point of view of a third party and input advice on the current situation, thereby allowing the users to learn active mental response skills.

In addition, a stress measurement method using pulse waves and autonomic nervous system activation could not detect a severe stress state (depression, suicide risk) of a user (including an emotional worker), but the system of the present invention can accurately diagnose a stress index (state, stress intensity, and duration) of the user by considering both a psychological test index through a psychological test as well as pulse wave measurement using a user terminal or an emotion index through the writing of an emotion diary.

In addition, the system of the present invention monitors the stress index (with the consent of the user), relieves stress by providing content corresponding to the stress index, and at the same time, transmits the stress index to the registered administrator or guardian terminal when the stress index exceeds a predetermined upper limit for a predetermined period, thereby managing the user's stress before reaching a dangerous state.

### DESCRIPTION OF DRAWINGS

FIG. 1 is a configuration diagram of a stress management system of the present invention.

FIG. 2 is a block diagram of a user terminal of the present invention.

FIG. 3 is a configuration diagram of a pulse wave signal extracting module 15.

FIG. 4 illustrates a process of an emotion diary module.

FIGS. 5 to 8 illustrate a screen in which the emotion diary module is executed by the user terminal.

### MODE FOR INVENTION

Hereinafter, preferred embodiments of the present invention will be described with reference to drawings. However, the scope of the present invention is not limited to the description or drawings of the following embodiments. That is, terms used in the present application are used only to describe specific embodiments, and are not intended to limit the present invention. A singular form may include a plural form if there is no clearly opposite meaning in the context. Further, it should be understood that term “include” or “have” described in the specification indicates that a feature, a number, a step, an operation, a component, a part or the combination thereof described in the specification is present, but does not exclude a possibility of presence or addition of one or more other features, numbers, steps, operations, components, parts or combinations thereof, in advance.

In addition, the terms “-er”, “-or” and “module” described in the specification mean units for processing at least one function and operation and can be implemented by hardware components or software components and combinations thereof.

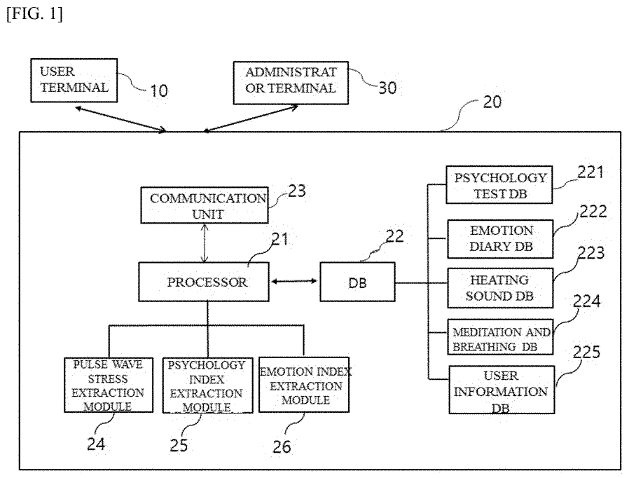

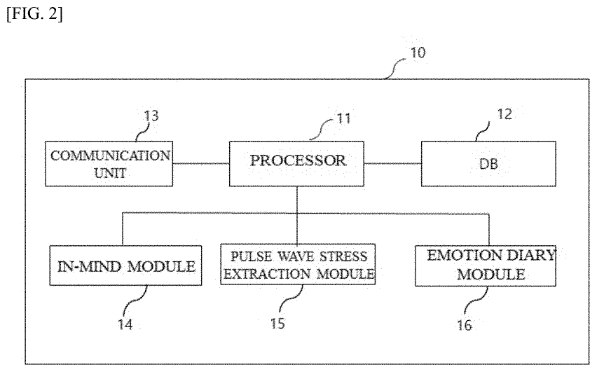

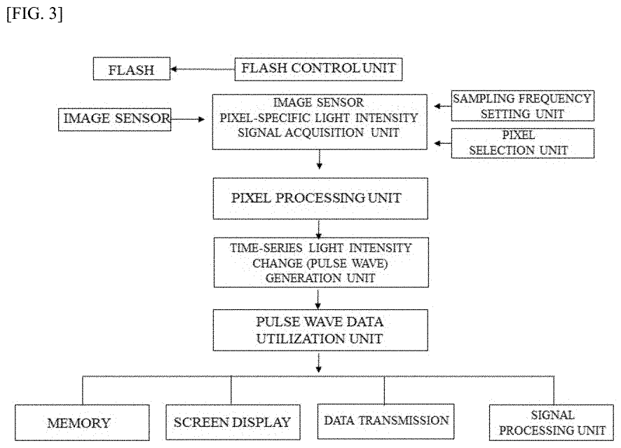

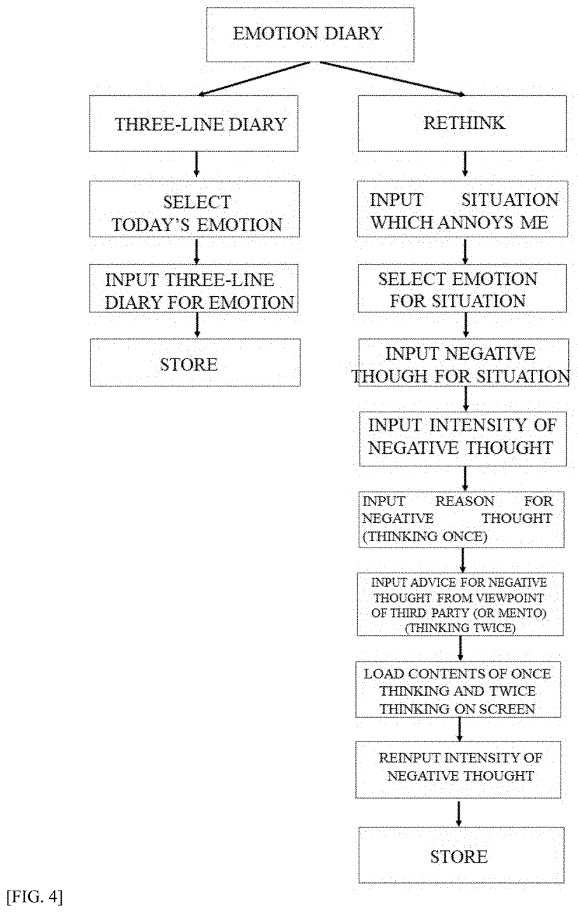

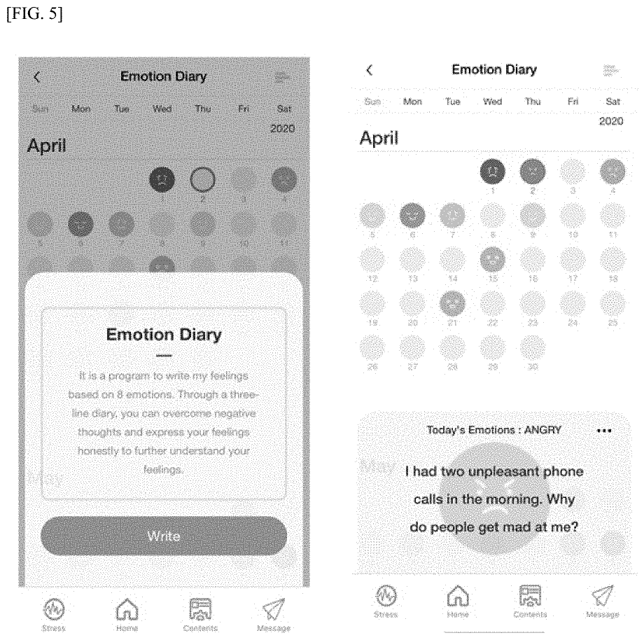

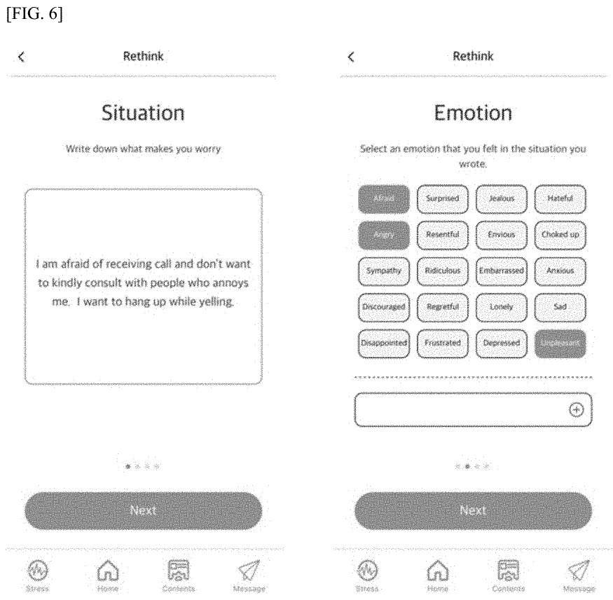

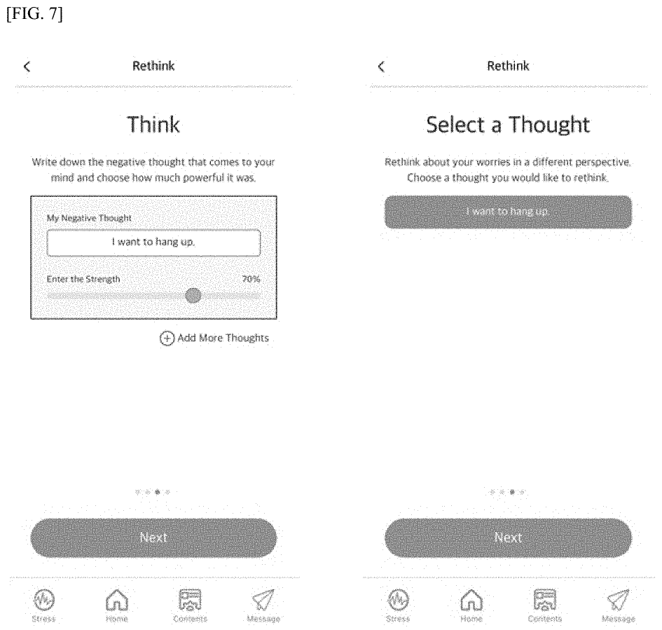

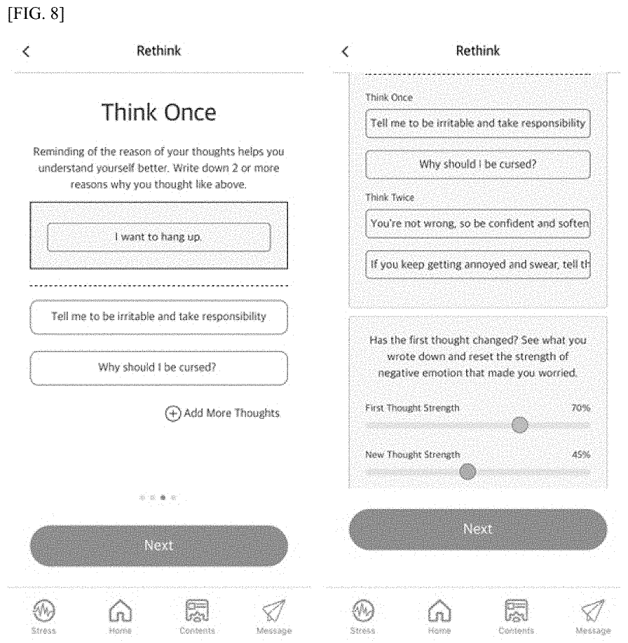

FIG. 1 is a configuration diagram of a stress management system of the present invention, FIG. 2 is a block diagram of a user terminal of the present invention, FIG. 3 is a configuration diagram of a pulse wave signal extracting module 15, FIG. 4 illustrates a process of an emotion diary module, and FIGS. 5 to 8 illustrate a screen in which the emotion diary module is executed by the user terminal.

Referring to FIG. 1, the system of the present invention includes a user terminal 10 and an in-mind server 20.

The user terminal 10 is a terminal capable of online access, including mobile and personal PCs, which may correspond to all personal terminals which may communicate with external devices such as smart phones, tablet PCs, slate PCs, notebook computers, digital broadcasting terminals, PDAs, PMPs, and navigation devices.

The user terminal 10 downloads and executes an in-mind application provided by the in-mind server.

The user terminal may include a processor 11, a DB 12, a communication unit 13, an in-mind module 14, a pulse wave signal extraction module 15, and an emotion diary module 16.

The processor 11 may process and execute the in-mind module to display recommended contents of the in-mind module, such as healing sounds, meditation techniques, breathing techniques, and the emotion diaries on the user terminal.

The processor 11 may process the pulse wave signal extraction module 15, and detect a pulse wave signal using a camera sensor and a built-in flash of the user terminal 10 and transmit the detected pulse wave signal to the server.

The server 20 may receive digital data of the pulse wave signal from the user terminal and calculate heart rate variability, autonomic nerve activity, and a pulse wave stress index.

For a method for measuring the pulse wave signal through the user terminal and the server and calculating the pulse wave stress index from signal data, a known method or Korean Patent Registration Nos. 10-0880392 and 10-0745972 may be referred to.

Referring to FIG. 3 (FIG. 2 refers to the contents of Korean Patent Registration No. 10-0880392), the pulse wave signal extraction module 15 may include a flash control unit, a light intensity signal acquisition unit, a pixel processing unit, a time series light intensity change generation unit, and a pulse wave data utilization unit.

The light intensity signal acquisition unit is a part that acquires a light intensity signal from an image sensor in chronological order, and here, detects a change in light intensity of a pixel of the image sensor for a specified pixel area set by a pixel selection unit at a time interval specified by a sampling frequency setting unit.

For example, for image data requiring information on the light intensity of one or more predetermined pixels, the light intensity of the i-th horizontal and j-th vertical pixel of the image sensor is defined as Intensity(i, j), and the pixel of the image sensor is subjected to discrete sampling by securing the light intensity signal in the processor 11. In addition, time-series data may be obtained by introducing a time index n and expressing Intensity (i, j, n) as a time axis.

Although the intensity (i, j, n) is a result of discretely detecting a direct pulse wave signal, a difference in the light intensity may occur for each pixel according to the arrangement of the finger, the flash, and the image sensor. That is, at horizontal point 1 and vertical point 2, the width of change in the light intensity may be large, and at horizontal point 50 and vertical point 30, the change in the light intensity may be small. In addition, in some pixels, a discriminating signal change may not be observed.

The pixel processing unit selects a pixel having a high signal intensity among a plurality of pixels. The pixel processing unit may more efficiently detect the pulse wave signal.

In the pixel processing unit, for example, a method for selecting a pixel having the largest change width in light quantity among several pixels is possible. In other words, this is a method for selecting a pixel that outputs the largest change by comparing the width of signal change between pixels. For example, if a pixel at horizontal point 100 and vertical point 20 has the largest change width in light intensity, discrete time series data of intensity (100, 20, n) may be used as the pulse wave signal. Alternatively, it is also possible to use all the pixels in which the light intensity is changed according to the light irradiation and the change in the amount of blood flow, and the average of the change in light intensity of all pixels may be used, and the discrete time series data thereof may be used as the pulse wave signal.

The time-series light intensity change generation unit may generate time-series data on an intensity of a pixel having the largest change width in light intensity, or may generate time-series data on the average of the light intensity change of all pixels.

The pulse wave data utilization unit may define the generated time series data as the pulse wave signal and transmit the generated time series data to the server 20.

The user terminal may receive a stress index and recommended content corresponding thereto from the server, and execute the recommended content in the application.

The in-mind server 20 may include a processor 21, a DB 22, a communication unit 23, a pulse wave stress index extraction module 24, a psychological index extraction module 25, and an emotion index extraction module 26.

The DB 22 may include a psychological test DB 221, an emotion diary DB 222, a healing sound DB 223, a meditation and breathing DB 224, and a user information DB 225.

The processor 21 may process the pulse wave stress index extraction module 25 and calculate the heart rate variability, the autonomic nerve activity, and the pulse wave stress index.

The pulse wave stress index extraction module 25 may calculate a heart rate per minute (BPM), heart rate variability, and sympathetic nerve activity based on the time series data of the pulse wave signal received from the user terminal.

For calculation of the heart rate per minute (BPM), the heart rate variability, and the sympathetic nerve activity, a known method may be used and contents of Korean Patent Registration No. 10-0745972 may be referred to.

The heart rate variability (HRV) means an interval between peaks of an electrocardiogram or pulse wave form and means a graph of a change in heart rate interval for a measured time.

The pulse wave stress index extraction module 25 may analyze a starting point, a peak point, and an overlapping pulse point through an interval of a heart rate variation and a change in capillary blood flow, and calculate the heart rate variability through the variation degree.

The pulse wave stress index extraction module 25 calculates a frequency domain parameter including LF activity indicating the sympathetic nerve activity through frequency analysis from the heart rate variability data. As an example of the calculation method, the pulse wave stress index extraction module may analyze a frequency domain from the heart rate variability data using fast Fourier transform.

The pulse wave stress index extraction module 25 may extract a power value of a low frequency band of 0.04 to 0.15 Hz, and calculate LF activity as a log value of the extracted power value of the low frequency band.

Further, the pulse wave stress index extraction module 25 may extract a power value of a high frequency band of 0.15 to 0.4 Hz, and calculate HF activity as a log value of the extracted power value of the high frequency band. The calculated LF activity reflects the sympathetic activity and the HF activity reflects parasympathetic nervous system activity.

The lower the LF activity in a standard range, the higher the HF activity in the standard range, the lower the stress.

The pulse wave stress index extraction module 25 may calculate a total power value representing values of the LF activity and the HF activity. The total power is an index obtained by the sum of the low frequency band to the high frequency band according to the fast Fourier transform. Therefore, the stress index may be calculated using the LF activity and the total power value.

For example, the pulse wave stress index extraction module 25 may determine the pulse wave stress index as follows based on the sympathetic nerve activity (minimum/maximum range 0 to 100 (average 50, standard deviation 10)).

When the sympathetic nervous system activity is less than 30, step 1 (stress is very low), when the sympathetic nervous system activity is more than 30 to less than 40, step 2 (low), when the sympathetic nervous system activity is more than 40 to less than 60, step 3 (normal), when the sympathetic nervous system activity is more than 60 to less than 70, step 4 (large), and when the sympathetic nervous system activity is 70 or more, step 5 (very large).

The server may provide the pulse wave stress index and recommended content corresponding thereto to the user terminal.

The recommended content may be any one of a healing sound, a meditation method, a breathing method, and an emotion diary.

The processor 21 of the in-mind server 20 may fetch the recommended content from the emotion diary DB, the healing sound DB, and the meditation and breathing DB, and transmit the recommended content to the user terminal.

In the user terminal, a real-time streaming image installed in the field may be executed together with the heating sound (recorded sound and image) selected from a category for region (Okinawa, Hawaii, Jeju Island, etc.) or for each topic (sea sounds, forest sounds, etc.).

The breathing method and the meditation method transmitted from the server may be performed on the user terminal, respectively.

The user terminal may execute an emotion diary content provided by the server.

Referring to FIGS. 4 to 8, the user terminal may process the emotion diary module 16 and load a three-line diary item and a rethinking item on the screen.

When the three-line diary item is executed, the emotion diary module 16 includes a screen loading step of selecting a today emotion among a plurality of examples (love, joy, anger, etc.) in the user terminal and a step of inputting contents (diary) related to the selected emotion into the user terminal.

When the rethink item is executed, the emotion diary module 16 may include a screen loading step in which a situation that annoys the user is input, a screen loading step in which an emotion that may express the situation is input, a screen loading step in which a negative though generated by the situation and the intensity of the thought are input, a screen loading step in which a reason for which the negative thought occurs, a screen loading step in which the user may input an advice for the negative though from the viewpoint of a mentor's position, and a screen loading step of re-inputting the intensity of the negative though again.

When the rethinking item is executed, the user may input a situation which annoys the user on the user terminal, input the emotion which may express the situation, input the negative though which occurs due to the situation and the intensity of the thought, input the reason for which the negative thought occurs, input the advice from the viewpoint of the mentor by the user, and input the strength of the negative though again.

In the present invention, the negative situation which occurs therefor is rethought from a third party (or mentor or objective gaze) and an execution screen capable of recording the negative situation is provided to objectify the negative situation felt by the user, thereby allowing an active mental response capability to be learned.

The user terminal may execute a psychological test sheet on the screen and input a current psychological state on a psychological test sheet screen.

The psychological test sheet includes a content capable of evaluating the stress as shown in Table 1.

TABLE 1

| | Not | Not | Nor- | | Very |
|---|---|---|---|---|---|
| Questionnaire content | at all | so | mal | So | so |
| 1. It's hard to get a proper rest because | 1 | 2 | 3 | 4 | 5 |
| my heart is uplifted. | | | | | |
| 2. I have a lot of feelings and can't | 1 | 2 | 3 | 4 | 5 |
| make a good decision. | | | | | |
| 3. It is difficult to concentrate on the | 1 | 2 | 3 | 4 | 5 |
| mind in a certain period of time. | | | | | |
| 4. I often struggle with indigestion. | 1 | 2 | 3 | 4 | 5 |
| 5. I am struggled with frequent | 1 | 2 | 3 | 4 | 5 |
| headaches. | | | | | |
| 6. I am easy to get excited. | 1 | 2 | 3 | 4 | 5 |

The processor 21 of the in-mind server 20 may process the psychological index extraction module 25 and calculate the psychological index of the user.

The psychological index extraction module 25 may include a step of summing a data input to the user terminal (a psychological test sheet answer value), and a step of calculating the psychological index by converting the sum value to a perfect score of 100 points.

The processor 21 of the in-mind server 20 may process the psychological index extraction module 26 and calculate the psychological index of the user.

The emotion index extraction module 26 may calculate the emotion index in consideration of the emotion input in the three-line diary, the emotion and negative thought intensity input in rethinking, and the negative thought intensity input in thinking twice.

1) The emotion index extraction module may grant 10 points when a positive word (love, joy, admiration, satisfaction) is selected among eight three-line emotions (love, joy, admiration, satisfaction, anger, annoyance, anxiety, and sadness) and −10 points when a negative word is selected.

2) The emotion index extraction module may grant 5 points when surprise, frown, and compassion are selected among 20 rethinking emotions (refer to Table 2), −10 points when fear, anger, resentment, anxiety, frustration, sadness, depression, frustration, and dislike are selected, and −5 points when the remaining words are selected.

TABLE 2
Fear, surprise, jealousy, disgust, anger, resentment, envy, frown,
compassion, embarrassment,
Shame, anxiety, frustration, regret, loneliness, sadness, upset, frustration,
depression, dislike

3) The emotion index extraction module may give −5 points, −10 points, and −15 points when the intensity of negative thoughts is 50 to 60%, more than 60 to 80%, and more than 80%, respectively.

4) The emotion index extraction module may grant 5 points, 10 points, and 15 points when the intensity of the negative thought input from thinking twice is reduced by 0 to 10%, by 10 to 30%, and by 30% or more, respectively.

On the other hand, the emotion index extraction module may uniformly grant −10 points when the intensity of the negative thought input in thinking twice is greater than the negative intensity in thinking once.

For example, if the user is in the state of 1) “worries” in the three-line diary, 2) “anxiety” in rethinking, 3) thought intensity is 70% 4) thought intensity is 50% in thinking twice,

the emotion index of the user is (−)10+(−)10+(−5)+10=−15 points.

As another example, if the user is in the state of 1) “satisfied” in the three-line diary, 2) “embarrassment” in rethinking, 3) thought intensity is 50% 4) thought intensity is 40% in thinking twice,

the emotion index of the user is 10+(−)5+(−5)+5=5 points.

The emotion index of the user may be in the range of (−)45 to 30.

The emotion index extraction module may convert the emotion index of the user (30 to (−)45) into 0 to 100 (converting the emotion index 45 to 100 and 30 to 0).

The server (process) may determine the pulse wave stress index as the stress index.

The server may calculate the stress index from the pulse wave stress index and the psychological test index.

The server may calculate the stress index from the pulse wave stress index and the emotion index.

The server may calculate the stress index from the pulse wave stress index, the emotion index, and the psychology index.

The server may calculate the stress index as follows.

Pulse wave stress index (0 to 100 converted points)×0.5+Psychology index (or emotion index)(0˜100 converted points)×0.5 1)

Pulse wave stress index (0 to 100 converted points)×0.4+Psychology index (0 to 100 converted points)×0.3+Emotion index (0 to 100 converted points)×0.3 2)

The server may provide the pulse wave stress index and recommended content corresponding thereto to the user terminal. For example, if the stress index is less than 30, healing sound content (50%) and the three-line diary (50%) in the emotion diary may be recommended, and if the stress index is 30 or more and less than 40, random (40%) of ASMR, the three-line diary (40%) in the three-line diary, and breathing method and meditation method (20%) may be recommended, and if the stress index is 40 or more and less than 60, breathing method and meditation method (40%), healing sound (40%), and random (20%) in the ASMR may be recommended, if the stress index is 60 or more and less than 70, content related to narcissism (40%), random (40%) in the ASMR, rethinking (20%) in the emotional diary may be recommended, and if the stress index is more than 70%, content related to narcissism (50%) and rethinking (50%) in the emotion diary may be recommended.

The server may transmit the stress index to a pre-registered manager terminal or guardian terminal when the stress index exceeds a predetermined upper limit value for a predetermined period or more.

For example, if the stress index exceeding 80 lasts for 7 days or more, the server may transmit the stress index to a registered manager terminal or guardian terminal.

The server of the present invention may manage the stress of the user before the user reaches a dangerous state by transmitting the stress index to the manager terminal or the guardian terminal.

The embodiments of the present invention disclosed in the present specification and drawings are provided only to provide specific examples to easily explain the technical content of the present invention and to aid understanding of the present invention, and are not intended to limit the scope of the present invention. In addition to the embodiments disclosed herein, it is apparent to those skilled in the art that other modified examples based on the technical spirit of the present invention can be executed.

1. A stress management system comprising:
a user terminal downloading and executing an in-mind application, detecting a pulse wave signal with a camera sensor and an in-built flash, and executing an emotion diary content and inputting an emotion state of a user; and
an in-mind server including a pulse wave stress index extraction module receiving digital data of the pulse wave signal from the user terminal and calculating heart rate variability, automatic nerve activity, and a pulse wave stress index and an emotion index extraction module calculating an emotion index from a received emotion state of the user, and calculating a stress index from the pulse wave stress index and the emotion index and providing a recommended content corresponding to the stress index to the user terminal,
wherein the user terminal receives the stress index and the recommended content corresponding thereto from the server, and execute the recommended content in the application,
the recommended content includes healing sounds, a meditation method, and a breathing method,
the user terminal executes a three-line diary item among the emotion diary contents, selects one or more of a plurality of emotion items for today's emotional state, and then records the selected emotion item,
the user terminal executes a rethink item among the emotion diary contents as follows to input and store the emotion state of the user,
the rethink item includes a screen loading step in which a situation that annoys the user is input, a screen loading step in which an emotion that may express the situation is input, a screen loading step in which a negative though generated by the situation and the intensity of the thought are input, a screen loading step in which a reason for the negative thought occurs, a (twice-thinking) screen loading step in which the user may input an advice for the negative though from the viewpoint of a mentor's position, and a screen loading step of re-inputting the intensity of the negative though again after the twice-thinking screen loading,
the emotion index extraction module calculates the emotion index in consideration of an emotion input in a three-line diary item, an emotion and a negative thought intensity input in a rethinking item, and the negative thought intensity input in the twice-thinking, and
the in-mind server stores a result of the emotion state selected and recorded by the user terminal in a DB and monitors the result.

2. The stress management system of claim 1, wherein the user terminal executes a psychology test,
the servers calculates a psychology test index of the user through psychology test data input into the user terminal, and
the server calculates the stress index from the pulse wave stress index, the motion index, and the psychology test index.

3. The stress management system of claim 1, wherein the server transmits the stress index to a pre-registered manager terminal or guardian terminal when the stress index exceeds a predetermined upper limit value for a predetermined period or more.
